(19) United States
(12) Patent Application Publication
(10) Pub. No.: US 2024/0053037 A1
(43) Pub. Date: Feb. 15, 2024

(54) AIR FILTER FOR PRODUCTION WORKSHOP

(71) Applicant: Kaitian ZHANG, Shandong Province (CN)

(72) Inventor: Kaitian ZHANG, Shandong Province (CN)

(21) Appl. No.: 17/906,590

(22) PCT Filed: Aug. 10, 2022
(86) PCT No.: PCT/CN2022/111428

§ 371 Date: Dec. 22, 2022

(51) Int. Cl. F24F 8/80; F24F 11/77; F24F 11/61; F24F 11/52; F24F 11/39; F24F 8/10; B01D 46/62; B01D 46/00; B01D 46/46; B01D 46/44; B01D 46/10; A61L 9/22; A61L 9/20

(52) U.S. Cl. CPC F24F 8/80; F24F 11/77; F24F 11/61; F24F 11/52; F24F 11/39; F24F 8/10; B01D 46/645; B01D 46/0032; B01D 46/0036; B01D 46/0049; B01D 46/46; B01D 46/0086; B01D 46/442; B01D 46/0028; B01D 46/10; A61L 9/22; A61L 9/20; F24F 2110/50

(57) ABSTRACT

An air filter including: a housing provided with an inner cavity, and an air inlet and an air outlet communicated with the inner cavity respectively; a filtration module provided in the inner cavity and located between the air inlet and the air outlet, and configured to filter the harmful substances; a fan provided in the inner cavity and located between the air inlet and the air outlet; a first detector configured to detect a first content of the harmful substances in the air in the production workshop; and a controller configured to control start, stop and rotational speed of the fan according to the first content. The air filter according to the embodiments of the present disclosure can realize intelligent and automatic filtration of the air in the production workshop, improve filtration efficiency and reduce energy consumption.

### CROSS-REFERENCE TO RELATED APPLICATIONS

This application is a National Stage of International Application No. PCT/CN2022/111428, filed Aug. 10, 2022, which is hereby incorporated by reference in its entirety.

### TECHNICAL FIELD

The present disclosure relates to the technical field of gas filtration, and particularly to an air filter for a production workshop.

### BACKGROUND

The air in the production workshop usually contains harmful substances that are not conducive to human health. For example, in the wood-processing workshop, a large amount of sawdust and dust will be produced during wood processing. Therefore, it is usually necessary to provide a dust removal device near a wood processing machine to absorb the sawdust and dust. However, some dust will still disperse in the air of the wood-processing workshop. Moreover, the air with the sawdust and dust is often mixed with volatiles of chemical products such as carpenter's glue and paint, such as formaldehyde, benzene and other harmful substances, which seriously affect the health of workers.

### SUMMARY

The embodiments of the present disclosure aim to provide an air filter to solve the problems pointed out in the Background or other similar problems.

The embodiments of the present disclosure provide an air filter for filtering harmful substances in air in a production workshop including: a housing provided with an inner cavity, and an air inlet and an air outlet communicated with the inner cavity respectively; a filtration module provided in the inner cavity and located between the air inlet and the air outlet, and configured to filter the harmful substances; a fan provided in the inner cavity and located between the air inlet and the air outlet; a detector configured to detect a content of the harmful substances in the air in the production workshop; and a controller configured to control start, stop and rotational speed of the fan according to the content.

The embodiments of the present disclosure have the following advantageous effects.

1. In the embodiments of the present disclosure, the controller controls the start, stop and rotational speed of the fan according to the content of the harmful substances detected by the detector, so as to realize real-time and automatic adjustment of the running state of the fan according to the change of the quality of the air in the production workshop, and realize intelligent and automatic air filtration, thus improving the air filtration efficiency, effectively improving the quality of the air in the production workshop, enhancing the safety of the production environment, while reducing the energy consumption.

2. In the embodiments of the present disclosure, the controller determines the air quality in real time according to the content of the harmful substances detected by the detector, and the display displays the air quality in real time, so that the workers can acquire the air quality in the production workshop in real time.

With reference to the following description and drawings, the specific embodiments of the present disclosure are disclosed in detail, and the ways in which the principles of the present disclosure can be adopted are pointed out. It should be appreciated that the embodiments of the present disclosure are not limited in scope. Within the scope of the spirit and clauses of the appended claims, the embodiments of the present disclosure include many changes, modifications and equivalents.

### BRIEF DESCRIPTION OF THE DRAWINGS

The drawings are included to provide a further understanding of the embodiments of the present disclosure, which constitute a part of the specification, illustrate the embodiments of the present disclosure, and together with the description, explain the principles of the present disclosure. Obviously, the drawings described below involve only some embodiments of the present disclosure. For those of ordinary skill in the art, other drawings can be derived from these drawings without any inventive efforts. In the drawings:

FIG. 1 and FIG. 2 are schematic diagrams of an assembly structure of an air filter according to an embodiment of the present disclosure;

FIG. 3 is an exploded structural diagram of an air filter according to an embodiment of the present disclosure;

FIG. 4 is a structural block diagram of an air filter according to an embodiment of the present disclosure;

FIG. 5 is a structural block diagram of an air filter according to another embodiment of the present disclosure.

### DETAILED DESCRIPTION

The foregoing and other features of the present disclosure will become apparent from the following description with reference to the drawings. In the description and drawings, specific embodiments of the present disclosure are specifically disclosed, which are some embodiments in which the principles of the present disclosure can be applied. It should be appreciated that the present disclosure is not limited to the described embodiments, and includes any modification, variation and equivalent that falls within the scope of the appended claims.

In the embodiments of the present disclosure, the terms such as ‘first’ and ‘second’ are used to distinguish different elements in terms of appellation, but they do not mean a spatial arrangement or a time sequence of these elements, and these elements should not be limited by these terms. The term ‘and/or’ includes any one or all combinations of one or more of the associated listed terms. The terms ‘comprise’, ‘include’ and ‘have’ refer to the presence of the stated features, elements, members or components, but do not exclude the presence or addition of one or more other features, elements, members or components.

The embodiments of the present disclosure provide an air filter for a production workshop that may be a wood-processing workshop or any other production workshop with harmful substances in the air. The harmful substances include particles such as sawdust and dust, and one or more kinds of harmful gases such as formaldehyde and benzene.

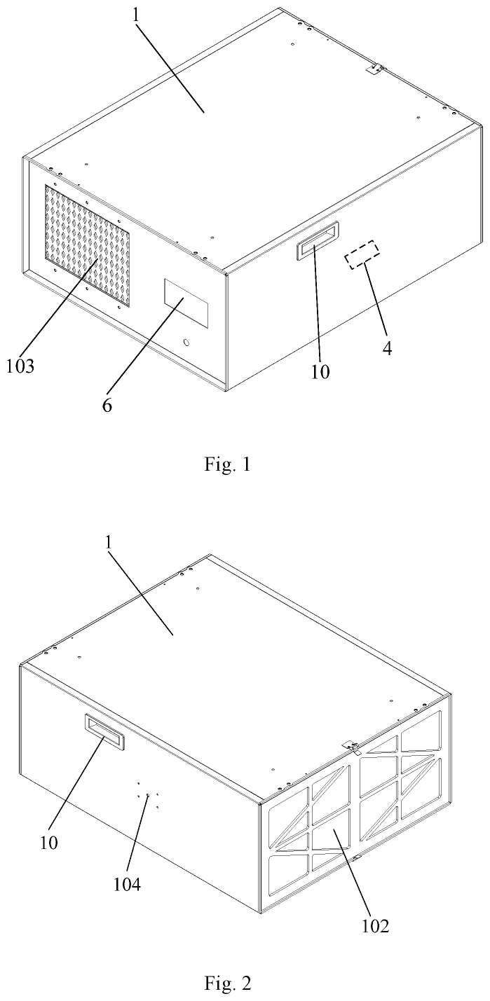

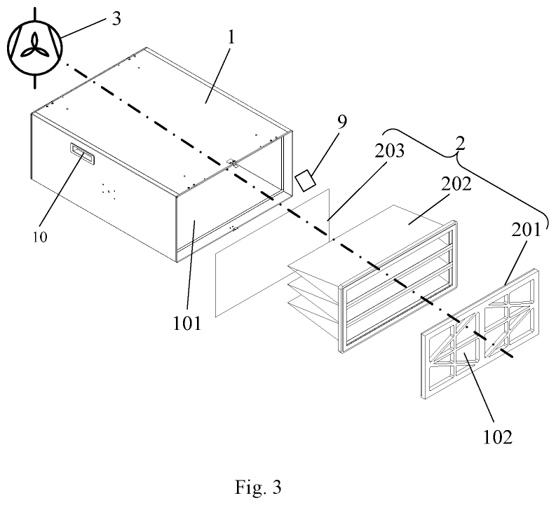

FIGS. 1 and 2 are schematic diagrams of assembly structures of an air filter according to an embodiment of the present disclosure. FIG. 3 is an exploded structural diagram of an air filter according to an embodiment of the present disclosure. As illustrated in FIGS. 1, 2 and 3, the air filter includes a housing 1, a filtration module 2, a fan 3, a first detector 4 and a controller 5. The housing 1 may be a box-type housing 1, with an inner cavity 101 therein, and an air inlet 102 and an air outlet 103 communicated with the inner cavity 101 on a sidewall of the housing 1. The air inlet 102 and the air outlet 103 may be respectively provided on two opposite sidewalls of the housing 1. The filtration module 2 is provided in the inner cavity 101 and located between the air inlet 102 and the air outlet 103, and configured to filter harmful substances in the air. The air to be filtered enters the inner cavity 101 through the air inlet 102 and is filtered by the filtration module 2 to become clean air, which is discharged through the air outlet 103. The fan 3 is provided in the inner cavity 101 and located between the air inlet 102 and the air outlet 103, and configured to provide power for driving the air to flow through the inner cavity 101. The fan 3 may be provided between the filtration module 2 and the air outlet to facilitate driving the air to flow through the filtration module 2.

The first detector 4 is configured to detect a content of harmful substances in the air in the production workshop (called as a first content). The controller 5 is configured to control start, stop and rotational speed of the fan 3 according to the first content, thus realizing the real-time and automatic adjustment of the running state of the fan 3 according to the real-time air quality in the production workshop, which can increase the air filtration efficiency, effectively improve the air quality in the production workshop, enhance the safety of the production environment, and reduce the energy consumption. The ‘content’ mentioned herein may be a concentration value, a mass fraction or a volume fraction.

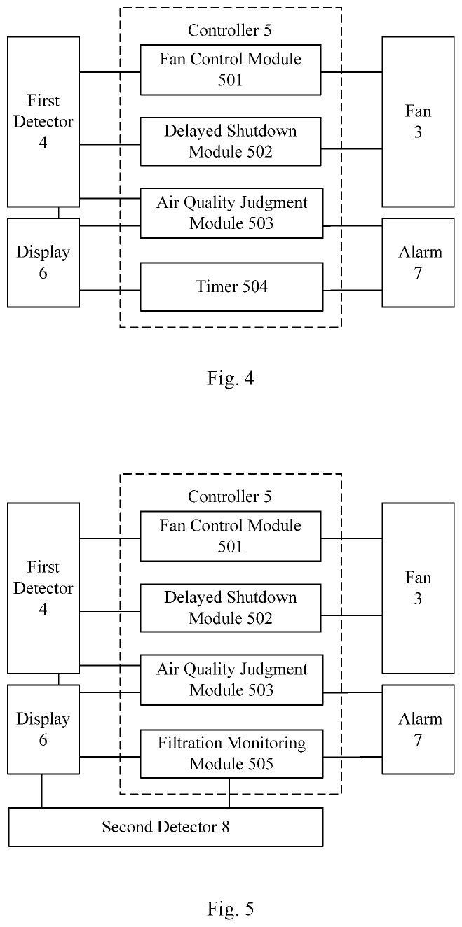

In some embodiments, as illustrated in FIG. 4, the controller 5 includes a fan control module 501 configured to compare the first content with a preset safety content, and control start, stop and rotational speed of the fan 3 according to a comparison result. If the comparison result is that the first content is less than or equal to the preset safety content, the fan control module 501 controls the fan 3 to stop; and if the comparison result is that the first content is greater than the preset safety content, the fan control module 501 controls the fan 3 to start and run at a rotational speed matched with the first content.

In one feasible technical solution, the fan control module 501 may determine the rotational speed of the fan 3 according to a preset corresponding relationship between the first content and the rotational speed of the fan 3, so as to control the fan 3 to run at a rotational speed matched with the first content. The preset corresponding relationship is between the harmful substance content and the rotational speed of the fan 3, and may be stored in the controller 5 in advance.

In another feasible technical solution, the fan control module 501 may determine the rotational speed of the fan 3 according to an actual content difference C1-C2 (calculated as ΔC) between the first content C1 and a preset safety content C2. Since the first content C1 is greater than the preset safety content C2, the difference therebetween is a positive value, and the rotational speed of the fan 3 rises as the difference ΔC increases. For example, if ΔC is greater than a preset content difference, the fan control module 501 controls the fan 3 to run at a first rotational speed; and if the actual content difference is less than or equal to the preset content difference, the fan control module 501 controls the fan 3 to run at a second rotational speed lower than the first rotational speed. Of course, it is also possible to set different preset content difference ranges according to actual needs, and set different rotational speeds, which may be a low speed, an intermediate speed and a high speed, corresponding to the different preset content difference ranges, respectively. The fan control module 501 controls the fan 3 to run at a rotational speed corresponding to the preset content difference range to which the actual content difference belongs.

In some embodiments, as illustrated in FIG. 4, the controller 5 includes a delayed shutdown module 502 configured to control the fan 3 for delayed shutdown when the fan 3 is in a running state and the first content is reduced to the preset safety content. In other words, when the fan 3 is in the running state and the first content is reduced to the preset safety content, the delayed shutdown module 502 controls the fan 3 to continue running for a preset time t and then shut down. The preset time t may be set according to actual needs and for example may be 10 seconds, 20 seconds or other time. The delayed shutdown module 502 enables the air filter of the present disclosure to have a delayed shutdown function.

Through researches, the inventor finds that when the fan 3 is in the running state, the air in the production workshop is continuously filtered by the air filter, and the content of harmful substances (i.e., the first content) contained therein continuously decreases. When the first content detected by the first detector 4 decreases to the preset safety content, the content of harmful substances in some area in the production workshop (such as the area far away from the first detector 4) is still higher than the preset safety content, and if the fan 3 shuts down immediately at this time, the air quality in such area is still poor. In this embodiment, by providing the delayed shutdown module 502 to control the fan 3 for delayed shutdown, the filtration of the air in the whole production workshop can be enhanced, thus improving the filtration effect of the whole production workshop.

In some embodiments, as illustrated in FIG. 4, the controller 5 includes an air quality judgment module 503 configured to judge an air quality in the production workshop in real time according to the first content. For example, the air quality judgment module 503 judges the air quality in the production workshop according to the comparison result between the first content and the preset safety content. If the first content is less than or equal to the preset safety content, it is judged that the air quality is qualified. If the first content is greater than the preset safety content, it is judged that the air quality is not qualified.

In some embodiments, as illustrated in FIGS. 1 and 4, the air filter further includes a display 6 communicatively connected to the controller 5, which transmits air quality data to the display 6 for displaying in real time, so that the workers in the production workshop can acquire the air quality in real time and make a timely response. For example, if the workers observe that the air quality displayed on the display 6 is not qualified, they can leave the production workshop temporarily to avoid injury. The display 6 may be a liquid crystal display screen.

A more detailed judgment criterion may also be set for the judgment of the air quality. For example, the air quality judgment module 503 determines the air quality according to the actual content difference ΔC between the first content C1 and the preset safety content C2 and a plurality of preset content difference ranges. For example, the plurality of preset content difference ranges are (−∞˜−A], (−A˜0], (0˜A] and (A˜+∞) in sequence, where A is a positive integer. If ΔC is within the range of (−∞˜−A], the air quality is judged to be excellent; if ΔC is within the range of (−A˜0], the air quality is judged to be good; if ΔC is within the range of (0˜A], the air quality is judged to be poor; and if ΔC is within the range of (A˜+∞), the air quality is judged to be extremely poor. By refining the judgment standard of the air quality, it is convenient to make a more targeted and effective response.

In order to remind the workers to make a timely response to the poor and/or extremely poor air quality, in one embodiment, the air filter may further include an alarm 7, which may be communicated with the controller 5 wiredly or wirelessly. When determining that the air quality is poor and/or extremely poor, the controller 5 sends an alarm signal to the alarm 7, and the alarm 7 emits alarm sound and/or flashes an alarm lamp to remind the workers to make a response upon receipt of the alarm signal.

In some embodiments, the controller 5 may include a timer 504 configured to collect accumulated running time of the fan 3, and determine remaining filtration time of the filtration module 2 according to the accumulated running time and a preset service life of the filtration module 2. The preset service life is the total filtration time of the filtration module 2, beyond which the filtration module 2 cannot effectively filter the air. For example, the timer 504 may be a countdown timer, with its starting time as the preset service life of the filtration module 2. The controller 5 may transmit data of the remaining filtration time to the display 6, and the display 6 displays the remaining filtration time of the filtration module 2 in real time, so that the workers can acquire the situation in real time and replace the filtration module 2 in time.

Optionally, as illustrated in FIG. 4, in order to remind the workers to make a timely response to an invalid state of the filtration module 2, the controller 5 sends an alarm signal to the alarm 7 when determining that the remaining filtration time of the filtration module 2 is zero, and the alarm 7 emits alarm sound and/or flashes an alarm lamp to remind the workers to make a response upon receipt of the alarm signal.

In some other optional embodiments, as illustrated in FIG. 5, the filtration device may include a second detector 8 configured to detect a content of harmful substances in the air filtered by the filtration module 2 (called as a second content). The controller 5 includes a filtration monitoring module 505 configured to determine a filtration state of the filtration module 2 according to the first content and the second content, so that the operator can make a response according to the filtration state. The controller 5 may transmit data of the filtration state to the display 6, and the display 6 displays the filtration state of the filtration module 2 in real time, so that the workers can acquire the situation in real time, and maintain or replace the filtration module 2 in time.

Specifically, the filtration monitoring module 505 compares the first content with the second content. If the second content is equal to or greater than the first content, the filtration monitoring module 505 determines that the filtration state of the filtration module 2 is an invalid state. If the second content is less than the first content, the filtration monitoring module 505 determines that the filtration state of the filtration module 2 is an effective state.

A more detailed evaluation criterion may also be set for the filtration state of the filtration module 2. For example, if the second content C3 is less than the first content C1, the filtration monitoring module 505 further evaluates the effective state of the filtration module 2 according to a difference C1-C3 (calculated as AM). If AM is less than a preset value, the filtration monitoring module 505 determines that the filtration state of the filtration module 2 is a low-efficiency state, and if AM is greater than the preset value, the filtration monitoring module 505 determines that the filtration state of the filtration module 2 is a high-efficiency state. If the filtration state of the filtration module 2 is the low-efficiency state, it may be considered to replace or clean the filtration module 2.

Optionally, as illustrated in FIG. 5, in order to remind the workers to make a timely response to the invalid state of the filtration module 2, the controller 5 transmits data of the filtration state of the filtration module 2 to the display 6 for displaying in real time, so that the workers in the production workshop can acquire the filtration state of the filtration module 2 in real time and make a timely response. For example, if the workers observe that the filtration state displayed on the display 6 is the low-efficiency state, the filtration module 2 may be replaced or cleaned.

Optionally, as illustrated in FIG. 5, in order to remind the workers to make a timely response to the invalid state of the filtration module 2, the controller 5 sends an alarm signal to the alarm 7 when determining that the filtration state of the filtration module 2 is the invalid state, and the alarm 7 emits alarm sound and/or flashes an alarm lamp to remind the workers to make a response upon receipt of the alarm signal. The alarm 7 may be integrated with the display 6 into one device.

In order to facilitate the workers to understand the concentration of harmful gases in the air in real time, as illustrated in FIG. 5, the first detector 4 and/or the second detector 8 may be communicatively connected to the display 6 wiredly or wirelessly, so as to transmit the first content and/or the second content to the display 6 for displaying.

In some embodiments, as illustrated in FIGS. 1 and 2, the first detector 4 is fixed on the housing 1 to detect the first content of harmful substances in the air in the production workshop. For example, the first detector 4 may be provided on an outer surface of the housing 1 or may be provided inside an opening 104 in the sidewall of the housing 1. The opening 104 may be provided adjacent to the fan 3, and a small amount of air outside the housing 1 enters the opening 104 by the negative pressure generated by the fan 3 and is detected by the first detector 4. Alternatively, the first detector 4 may be provided at the air inlet 102. Of course, the first detector 4 may not be provided on the housing 1, but provided independently of the housing 1, and even provided at a position far away from the housing 1, as long as the content of harmful substances in the air in the production workshop can be detected. The first detector 4 may be communicatively connected to the controller 5 wiredly or wirelessly, so that the first content detected by the first detector 4 may be sent to the controller 5.

The first detector 4 may include a dust sensor configured to detect a concentration of particles in unfiltered air, and/or a gas sensor configured to detect a concentration of harmful gases in unfiltered air. The gas sensor may be an electrochemical gas sensor. The dust detector and the gas sensor may convert data of the detected content into electrical signals and send the electrical signals to the controller 5. The types of components of the first detector 4 may be determined according to the compositions of the harmful substances to be detected.

The dust sensor may detect a concentration of particles with a particle diameter of not less than 0.03 μm in the air, such as fume, dust, wood powder, welding slag, etc. The electrochemical gas sensor may detect a concentration of chemical gases in the air, such as nitric oxide, nitrogen dioxide, formaldehyde and benzene.

In some embodiments, the second detector 8 is provided in the inner cavity 101 of the housing 1 and located between the filtration module 2 and the air outlet 103. In other words, in an air flow direction, the second detector 8 is located downstream of the filtration module 2 to detect the second content of harmful substances in the air that has been filtered by the filtration module 2. For example, the second detector 8 may be provided at the air outlet or on the inner surface of the housing 1. The second detector 8 may be communicatively connected to the controller 5 wiredly or wirelessly, so that data of the content of harmful substances detected by the second detector 8 can be transmitted to the controller 5.

The second detector 8 may include a dust sensor configured to detect a concentration of particles in the filtered air, and/or a gas sensor configured to detect a concentration of harmful gases in the filtered air. The gas sensor may be an electrochemical gas sensor. The types of components of the second detector 8 may be determined according to the compositions of harmful substances to be detected. It can be appreciated that the component types of the second detector 8 and the first detector 4 may be consistent, so as to compare the contents of the same harmful substance before and after filtration.

In some embodiments, in order to improve the filtration effect while reducing the energy consumption, the controller 5 may be configured to be in a standby state after the fan 3 shuts down, and the first detector 4 is configured to continuously detect the content of harmful substances. Therefore, when the fan 3 is in the shutdown state and the controller 5 is in the standby state, the first detector 4 can transmit the detected first content to the controller 5 in real time, and once the first content exceeds the preset safety content, the controller 5 is immediately awakened to control the fan 3 to start immediately.

In some embodiments, as illustrated in FIG. 3, the air filter further includes a disinfection and sterilization module 9 provided in the inner cavity 101 of the housing 1 and located between the filtration module 2 and the air outlet 103. In other words, in the air flow direction, the sterilization module 9 is located downstream of the filtration module 2, and configured to disinfect and sterilize clean air obtained by filtration of the filtration module 2 to further purify the air, which is especially suitable for a production workshop with a high air quality requirement, and/or a production workshop which is dark, damp and poorly ventilated. The disinfection and sterilization module 9 may include at least one selected from a plasma generator and an ultraviolet irradiation lamp.

In some embodiments, as illustrated in FIG. 3, the filtration module 2 includes a primary filtration layer 201, a medium efficiency filtration layer 202, and a high efficiency filtration layer 203 with progressively increasing filtration accuracies and sequentially provided along an air flow direction in the inner cavity. The primary filtration layer 201 may be composed of metal meshes and textile fabrics, which have larger apertures and can filter particles with larger particle diameters in the air to prevent the particles from blocking the medium efficiency filtration layer and the high efficiency filtration layer. The primary filtration layer 201 may be fixed on a side surface of the support plate of the housing 1, and the air inlet 102 may be provided on the support plate. The medium efficiency filtration layer 202 may be composed of electrostatic meshes, which can ionize the air. The high efficiency filtration layer 203 may be composed of a high-density breathable material, which can effectively absorb the dust and harmful gases in the ionized air.

When the air filter is in operation, under the negative pressure generated by the fan 3 in operation, the air in the production workshop enters the inner cavity 101 from the air inlet 102, sequentially flows through the primary filtration layer, the medium efficiency filtration layer and the high efficiency filtration layer for filtrations in turn, then flows through a plasma generator and/or an ultraviolet irradiation lamp for being disinfected and sterilized into clean air, and flows back to the production workshop through the air outlet 103. The clean air circulates in the production workshop, thus realizing the purification and sterilization of the air in the whole production workshop.

In some embodiments, as illustrated in FIG. 2, the outer surface of the housing 1 is provided with an inward concave grip portion 10, into which a finger can insert to facilitate manual handling of the air filter.

The preferred embodiments of the present disclosure are described above with reference to the drawings. Many features and advantages of these embodiments will be clear from the detailed description, so the appended claims are intended to cover all the features and advantages of these embodiments that fall within their true spirit and scope. In addition, since many modifications and changes are easily conceivable for those skilled in the art, it is not intended to limit the embodiments of the present disclosure to the precise structures or operations illustrated and described, but to cover all suitable modifications and equivalents falling within the scope.

1. An air filter for filtering harmful substances in air in a production workshop, comprising:
a housing provided with an inner cavity, and an air inlet and an air outlet communicated with the inner cavity respectively;
a filtration module provided in the inner cavity and located between the air inlet and the air outlet, and configured to filter the harmful substances;
a fan provided in the inner cavity and located between the air inlet and the air outlet;
a detector configured to detect a content of the harmful substances in the air in the production workshop; and
a controller configured to control start, stop and rotational speed of the fan according to the content.

2. The air filter according to claim 1, wherein the controller comprises:
a fan control module configured to compare the content with a preset safety content, and control the start and stop of the fan according to a comparison result.

3. The air filter according to claim 2, wherein the fan control module is further configured to determine the rotational speed of the fan according to a preset corresponding relationship between the content and the rotational speed of the fan, and control the fan to run at the determined rotational speed.

4. The air filter according to claim 1, wherein the controller further comprises:
a delayed shutdown module configured to control the fan for delayed shutdown when the fan is in a running state and the content is reduced to a preset safety content.

5. The air filter according to claim 1, wherein,
the controller comprises an air quality judgment module configured to judge an air quality of the air in the production workshop according to the content;
the air filter comprises a display communicatively connected to the controller to display the air quality.

6. The air filter according to claim 1, wherein the controller comprises:
a timer configured to collect accumulated running time of the fan and determine remaining filtration time of the filtration module according to the accumulated running time and a preset service life of the filtration module;
the air filter comprises a display communicatively connected to the timer to display the remaining filtration time.

7. The air filter according to claim 1, wherein the detector is provided on the housing.

8. The air filter according to claim 1, wherein the harmful substances comprise particles and/or harmful gases; the detector comprises:
a dust sensor configured to detect a concentration of the particles; and/or,
a gas sensor configured to detect a concentration of the harmful gases.

9. The air filter according to claim 1, further comprising:
a disinfection and sterilization module provided in the inner cavity and located between the filtration module and the air outlet, and configured to disinfect and sterilize the air that has been filtered by the filtration module.

10. The air filter according to claim 9, wherein,
the disinfection and sterilization module comprises at least one selected from a plasma generator and an ultraviolet irradiation lamp.

11. The air filter according to claim 1, wherein,
the filtration module comprises a primary filtration layer, a medium efficiency filtration layer and a high efficiency filtration layer which are sequentially provided along an air flow direction.
